SEQUENCE LISTING (4 sequences)

SEQ ID NO: 1 - DNA, 1353 bp, Artificial Sequence
  Antibody 2D03 Heavy Chain
gaggtgcagctgttggagtctgggggaggcttggtacagcctggggggtccctgagactctcctgtgcagcctctggattcaccttcagtaacgcctggatgagctgggtccgccaggctccagggaaggggctggagtgggtctcaagtattagtgttggtggacataggacatattatgcagattccgtgaagggccggtccaccatctccagagacaattccaagaacacgctgtatctgcaaatgaacagcctgagagccgaggacactgccgtgtattactgtgcacggatacgggtgggtccgtccggcggggcctttgactactggggccagggtacactggtcaccgtgagctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 2 - DNA, 648 bp, Artificial Sequence
  Antibody 2D03 Light Chain
cagtctgtgctgactcagccaccctcagcgtctgggacccccgggcagagggtcaccatctcctgctctggaagcaacaccaacattgggaagaactatgtatcttggtatcagcagctcccaggaacggcccccaaactcctcatctatgctaatagcaatcggccctcaggggtccctgaccgattctctggctccaagtctggcacctcagcctccctggccatcagtgggctccggtccgaggatgaggctgattattactgtgcgtcatgggatgccagcctgaatggttgggtattcggcggaggaaccaagctgacggtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 3 - protein, 451 aa, Artificial Sequence
  Antibody 2D03 Heavy Chain translated sequence
EVQLLESGGGLVQPGGSLRLSCAASGFTFSNAWMSWVRQAPGKGLEWVSSISVGGHRTYYADSVKGRSTISRDNSKNTLYLQMNSLRAEDTAVYYCARIRVGPSGGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 216 aa, Artificial Sequence
  Antibody 2D03 Light Chain translated sequence
QSVLTQPPSASGTPGQRVTISCSGSNTNIGKNYVSWYQQLPGTAPKLLIYANSNRPSGVPDRFSGSKSGTSASLAISGLRSEDEADYYCASWDASLNGWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

(19) United States
(12) Patent Application Publication
(10) Pub. No.: US 2011/0014203 A1
(43) Pub. Date: Jan. 20, 2011

(54) FORMULATION

(76) Inventors: Fredrik Nilsson, Malmo (SE); Karl Johan Brink, Genarp (SE)

Correspondence Address: FISH & RICHARDSON P.C. (TC), PO BOX 1022, MINNEAPOLIS, MN, 55440-1022, US

(21) Appl. No.: 12/810,906

(22) PCT Filed: Dec. 22, 2008
(86) PCT No.: PCT/EP2008/011043

§ 371 Date: Oct. 5, 2010

Related U.S. Application Data
(60) Provisional application No. 61/017,290, filed on Dec. 28, 2007.

(51) Int. Cl. A61K 39/395; A61P 9/10; A61M 5/178

(52) U.S. Cl. 424/142.1; 604/187

(57) ABSTRACT

A stable, aqueous pharmaceutical composition comprising an antibody having a heavy chain amino acid sequence of SEQ ID No: 3 and a light chain amino acid sequence of SEQ ID No: 4 and a pharmaceutically-acceptable adjuvant, diluent, carrier or excipient, wherein said composition has a pH of 4 to 6.

The present invention relates to pharmaceutical compositions, and in particular to stable, aqueous pharmaceutical compositions of an antibody that binds oxidised LDL that is useful for the treatment of atherosclerosis.

Atherosclerosis is a multifactorial disease developing preferentially in subjects presenting biochemical risk factors including smoking, hypertension, diabetes mellitus, hypercholesterolemia, elevated plasma low-density lipoprotein (LDL) and triglycerides, hyperfibrinogenemia and hyperglycemia. Atherosclerosis is a chronic disease that causes a thickening of the innermost layer (the intima) of large and medium-sized arteries. It decreases blood flow and might cause ischemia and tissue destruction in organs supplied by the affected vessel. Atherosclerotic lesions develop over a number of decades in humans, leading to complications such as coronary and cerebral ischemic and thromboembolic diseases and myocardial and cerebral infarction.

Atherosclerosis is the major cause of cardiovascular disease including acute myocardial infarction, stroke and peripheral artery disease. Cardiovascular disease is the leading cause of morbidity and mortality in industrialised countries and progresses steadily in emerging countries, with coronary atherosclerosis being the main underlying pathology. Current therapy of atherosclerosis is not completely effective at preventing disease development and complication.

The disease is initiated by accumulation of lipoproteins, primarily LDL, in the extracellular matrix of the vessel. These LDL particles aggregate and undergo oxidative modification. Oxidised LDL is toxic and causes vascular injury. Atherosclerosis represents, in many respects, a response to this injury including inflammation and fibrosis.

High plasma levels of cholesterol, and in particular high levels of LDL, are generally recognised as driving forces for development of atherosclerosis whereas high levels of high-density lipoprotein (HDL) counteract development of atherosclerosis. HDL has consequently been called the good cholesterol while LDL has been called the bad cholesterol. Simplified, LDL transports cholesterol to tissue while HDL absorbs cholesterol from tissue and transports it to the liver where it becomes degraded. Therapeutic strategies to reduce LDL and increase HDL are under development for treatment of atherosclerosis.

ApoB-100 is the protein component of LDL which is the main carrier of cholesterol in human serum. Oxidation of LDL is an essential step in its conversion to an atherogenic particle and the oxidative modifications drive the initial formation of fatty streaks, the earliest visible atherosclerotic lesion.

Radiolabelled forms of antibodies that bind to oxidised LDL can also be used for radioimmunodetection of atherosclerotic lesions in experimental animals (Tsimikas et al, 2000). An ¹²⁵Iodine-labelled anti-MDA lysine epitope antibody was used to detect plaque in mice and rabbits, and the injected antibody was found to localise to plaques in the aorta.

Human antibodies have been developed from a recombinant antibody fragment library called n-CoDeR® that were directed against oxidised peptides derived from human ApoB-100 (WO 02/080954). These recombinant antibodies, as well as antibodies against other oxidised LDL epitopes, were shown to significantly inhibit plaque formation and prevent the development of atherosclerotic lesions in animal models (Schiopu et al, 2004; WO 2004/030607; U.S. Pat. No. 6,716,410).

Subsequently, the antibodies that bind to oxidised ApoB-100 disclosed in WO 2004/030607, especially IEI-E3, LDO-D4, KTT-B8 and 2-D03, were shown to actively induce the regression of pre-existing, established atherosclerotic plaques in the aorta after a few weeks of treatment (WO 2007/025781). Such antibodies have been suggested for therapy of advanced atherosclerosis to revert disease progression resulting in a reduced plaque burden, as well as for therapy of cardiovascular diseases associated with atherosclerosis. Thus, targeting oxidised LDL with monoclonal antibody therapy is an increasingly attractive treatment modality for some of the leading causes of death in the Western world.

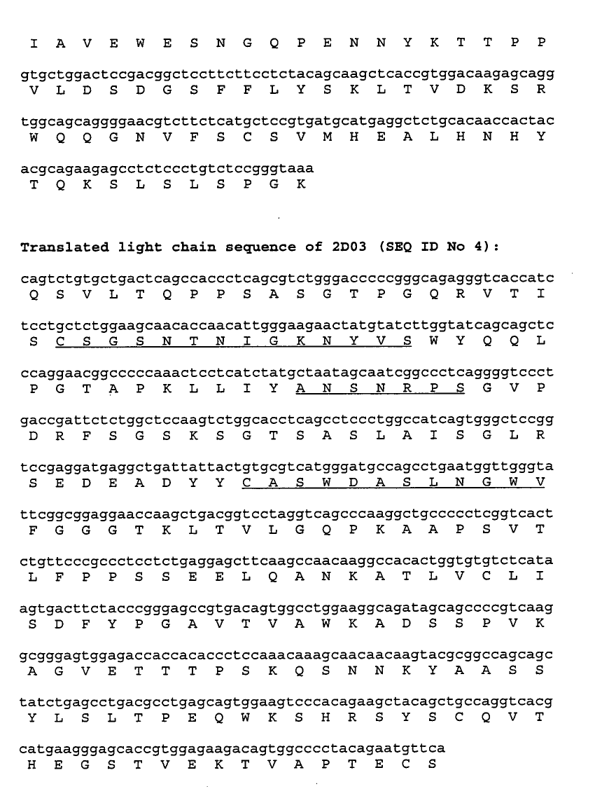

The antibody in WO 2007/025781 which was most efficacious at inducing the regression of pre-existing plaques was antibody 2D03. The V_{H} and V_{L} sequences of antibody 2D03 are given in FIG. 3 of WO 2004/030607, and the CDR sequences of antibody 2D03 are listed in Table 2 of WO 2007/025781. As is well known in the art, a stable formulation simplifies drug distribution and storage, thereby reducing costs to both the pharmaceutical industry and the patient (Lucas et al (2004) Pharmaceutical Technology, July 2004 Issue, pp: 69-72). There is a need in the art for a stable pharmaceutical formulation comprising antibody 2D03 which is suitable for therapeutic use.

In the past several years, advances in biotechnology have made it possible to produce a variety of proteins, such as antibodies, for pharmaceutical applications using recombinant DNA techniques. Because proteins are larger and more complex than traditional organic and inorganic drugs (i.e. possessing multiple functional groups in addition to complex three-dimensional structures), the stable formulation of such proteins poses special problems. For a protein, such as an antibody, to remain biologically active, a formulation must preserve intact the conformational integrity of at least a core sequence of the protein's amino acids while at the same time protecting the protein's functional groups from degradation. Degradation pathways for proteins can involve chemical instability (i.e. any process which involves modification of the protein by bond formation or cleavage resulting in a new chemical entity) or physical instability (i.e. changes in the higher order structure of the protein). Chemical instability can result from deamidation, racemization, hydrolysis, oxidation, beta elimination or disulfide exchange. Physical instability can result from denaturation, aggregation, precipitation or adsorption, for example. The three most common protein degradation pathways are protein aggregation, deamidation and oxidation (Cleland et al (1993) Critical Reviews in Therapeutic Drug Carrier Systems 10(4): 307-377).

A number of formulations are known to enhance the stability of antibody compositions. For example, U.S. Pat. No. 6,171,586 describes a formulation which contains a polyol and a surfactant which act to stabilise the antibody, and does not contain sodium chloride.

Surprisingly and unexpectedly, we have found that antibody 2D03 displays different solubility characteristics from previous n-CoDeR® generated antibody products. Typically, a 10-20 mM phosphate buffer, containing 150 mM Nacl, pH 7-7.5, results in a stable formulation of n-CoDeR® generated antibody products.

In contrast to other antibodies with the n-CoDeR® framework, we have found that 2D03 aggregates at pH above 6.0. Since a pH of below 4 is unsuitable for a composition for intravenous or subcutaneous administration to a patient, we have identified a narrow pH window for a composition containing antibody 2D03 which has a useful period of stability on storage, and which is suitable for intravenous or subcutaneous administration to a patient.

In addition, concentration or buffer exchange by ultrafiltration of 2D03 generated aggregates when performed in solutions with low conductivity (less than 100 mM NaCl equivalent). Initial studies showed that the antibody 2D03 product could be concentrated to at least 160 mg/ml if the pH was maintained at pH 5.5 and 150 mM NaCl was included.

In initial trials, attempts to increase the stability of 2D03 by addition of standard additives such as polysorbate 20, arginine, histidine, glutamic acid and mannitol were not sufficiently effective.

Accordingly, a first aspect of the invention thus provides an aqueous pharmaceutical composition comprising antibody 2D03 and a pharmaceutically-acceptable adjuvant, diluent, carrier or excipient, wherein the composition has a pH of 4 to 6.

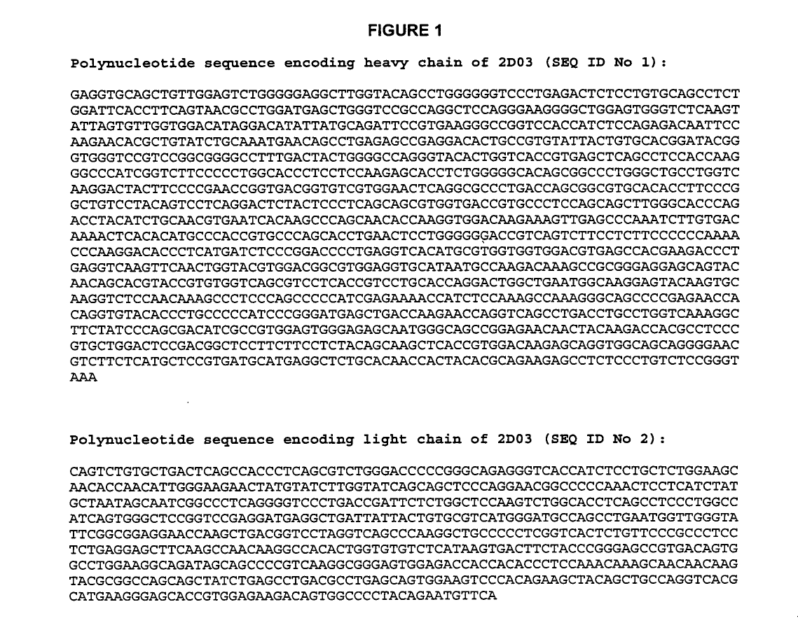

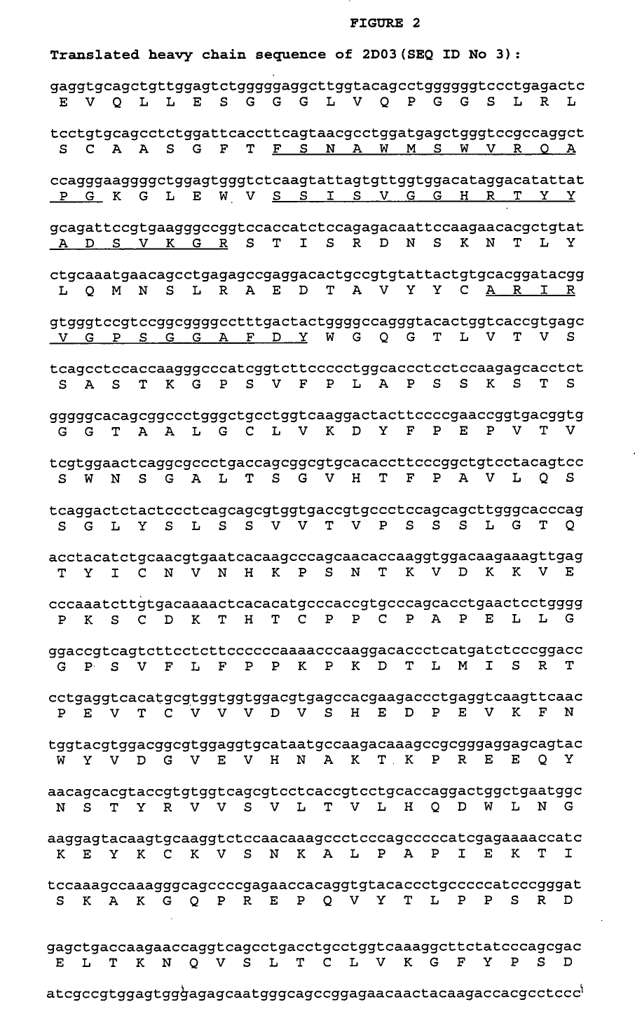

2D03 is a fully human monoclonal IgG, antibody directed against oxidised LDL. The polynucleotide sequences encoding the heavy chain and light chain of antibody 2D03 are given in FIG. 1 and have been assigned SEQ ID No: 1 and SEQ ID No: 2, respectively. The amino acid sequences of the heavy chain and light chain of antibody 2D03 are given in FIG. 2 and have been assigned SEQ ID No: 3 and SEQ ID No: 4, respectively.

Accordingly, this aspect of the invention provides an aqueous pharmaceutical composition comprising an antibody having a heavy chain amino acid sequence of SEQ ID No: 3 and a light chain amino acid sequence of SEQ ID No: 4 and a pharmaceutically-acceptable adjuvant, diluent, carrier or excipient, wherein the composition has a pH of 4 to 6.

The antibody in the pharmaceutical composition may be prepared using any of the techniques well known in the art for generating antibodies. Exemplary methods for producing recombinant antibodies are described in more detail below.

The term “pharmaceutical composition” is well known in the art and refers to a preparation which is in such form as to permit the biological activity of the active ingredient (i.e. the antibody 2D03) to be effective, and which contain no additional components which are toxic to the patients to whom the composition would be administered. Pharmaceutically acceptable adjuvants, diluents, carriers and excipients are those which can reasonably be administered to a patient to provide an effective dose of the active ingredient employed, and are well known in the art.

There are a number of ways by which the stability of a pharmaceutical composition comprising an antibody may be evaluated, several of which are detailed below in Examples 2 and 3. For example, the stability of the antibody-containing pharmaceutical composition may be determined by assessing its purity, e.g. by size-exclusion chromatography, by cation-exchange chromatography and/or by SDS-PAGE. Additionally or alternatively, the stability of the antibody-containing pharmaceutical composition may be determined by its appearance whether evaluated by eye or by light scattering at 410 nm. Further additionally or alternatively, the stability of the pharmaceutical composition may be determined by reference to the activity of antibody 2D03, and typically by reference to the antigen binding activity of antibody 2D03 (e.g. the ability to bind to MDA-ApoB100). Thus by a “stable” pharmaceutical composition, we mean that the antibody maintains at least 50% of its ability to bind to MDA-ApoB100 after storage (for the specified times and under the specified conditions), in comparison to an antibody that has not been stored as defined. More preferably, the antibody maintains at least 60%, or at least 70%, 80%, 90% or 95% of its ability to bind to MDA-ApoB100. Still more preferably the antibody maintains at least 99%, or 100%, of its ability to bind to MDA-ApoB100 after storage as defined for the stated times and under the conditions given below.

By a “stable pharmaceutical antibody preparation”, we include the meaning that the purity of the antibody preparation after storage as defined for the stated times and under the conditions given below is at least 90% of intact monomeric antibody, more preferably 95% or more of intact monomeric antibody, specifically 96% or 97% 98% or 99% or more of intact monomeric antibody. Preferably, the purity of the antibody preparation after storage is determined and/or measured using size exclusion chromatography, as described in the accompanying Examples.

As described herein, the pharmaceutical composition of this aspect of the invention is a stable pharmaceutical composition. Typically, the composition is stable at a storage temperature of about 2-8° C. for at least 4 weeks. Advantageously, the pharmaceutical composition is stable at about 2-8° C. for at least 8 weeks. Yet more preferably, the pharmaceutical composition is stable at about 2-8° C. for at least 14 weeks, or more. Even more preferably, the pharmaceutical composition is stable at about 2-8° C. for at least 12 months, conveniently at least 1.5 years, and advantageously at least 3 years. More advantageously the pharmaceutical composition is stable for at least 4 or 5 years. Typically, the pharmaceutical composition is stable following freezing and thawing of the composition.

Suitably, the pharmaceutical composition is stable at about 24° C. (for example, at 25° C.) for at least 4 weeks, and more preferably for at least 8 weeks, or more. As demonstrated in the accompanying Examples, pharmaceutical compositions of the invention may be stable for 6 months at 25° C.

In embodiments of the invention, the pharmaceutical composition has a minimum pH of 4.1, or 4.2, or 4.3, or 4.4, or 4.5, or 4.6, or 4.7, or 4.8, or pH 4.9, and a maximum pH of 6.0.

In other embodiments, the pharmaceutical composition has a maximum pH of 5.9, or 5.8, or 5.7, or 5.6, or 5.5, or 5.4, or 5.3, or 5.2, or 5.1, and a minimum pH of 4.

In still other embodiments, the pharmaceutical composition has a maximum pH of 5.9, or 5.8, or 5.7, or 5.6, or 5.5, or 5.4, or 5.3, or 5.2, or 5.1, and a minimum pH of 4.5

In one embodiment, the pharmaceutical composition has a pH of 4.9 to 5.1, and more specifically a pH of 5.0.

In another embodiment, the pharmaceutical composition has a pH of 5 to pH 6, for example pH 5.0 to 5.9, pH 5 to 5.8, pH 5 to 5.7, or pH 5.0 to 5.6. In a more specific embodiment the pharmaceutical composition has a pH of 5.4 to 5.6, and more specifically a pH of about 5.5.

As will be appreciated, in order to maintain the desired pH, the pharmaceutical composition comprises a buffer. As used herein, “buffer” refers to a buffered solution that resists changes in pH by the action of its acid-base conjugate components. The buffer of this invention has a pH in the range from about 4 to about 6; preferably from about 4.5 to about 5.8; more preferably from about 4.8 to about 5.6; and most preferably has a pH of between 5.0 and 5.6. Examples of buffers that control the pH in this range include acetate (e.g. sodium acetate), succinate (such as sodium succinate), gluconate, citrate and other organic acid buffers. Preferably, the buffer is not a phosphate buffer, especially where a freeze-thaw stable formulation is desired.

The buffer concentration can be from about 1 mM to about 50 mM, suitably from about 5 mM to about 40 mM, and preferably from about 10 mM to about 30 mM, depending, for example, on the buffer and the desired isotonicity of the formulation.

In a preferred embodiment, the pharmaceutical composition comprises an acetate buffer. Typically, the acetate is present at 5-30 mM, and more preferably at 10-30 mM. In a more specific embodiment, the acetate is present at about 20 mM.

In an embodiment, the pharmaceutical composition further comprises sodium chloride. The sodium chloride may be present from about 50 mM to about 200 mM, suitably from about 100 mM to about 200 mM, and preferably at about 150 mM.

Additionally or instead of sodium chloride, the pharmaceutical composition may further comprise other salts and/or amino acids.

Typically, the antibody is present in the pharmaceutical composition at a concentration of 10 to 200 mg/ml, for example 25 to 150 mg/ml. In specific embodiments, the antibody is present at 25±10 mg/ml, 120±20 mg/ml, or about 150±10 mg/ml in the pharmaceutical composition. For example, the antibody may be present in the pharmaceutical composition at a concentration of 10 mg/ml or 20 mg/ml or 30 mg/ml or 40 mg/ml or 50 mg/ml or 60 mg/ml or 70 mg/ml or 80 mg/ml or 90 mg/ml or 100 mg/ml or 110 mg/ml or 120 mg/ml or 130 mg/ml or 140 mg/ml or 150 mg/ml or 160 mg/ml. Preferably, the antibody is present in the pharmaceutical composition at a concentration of less than 160 mg/ml; such as less than 100 mg/ml or less than 50 mg/ml or less than 25 mg/ml.

A preferred embodiment of the invention provides a stable, aqueous pharmaceutical composition comprising, per ml:
15 to 160 mg of antibody 2D03 as defined above;
8.77 mg sodium chloride;
2.35 mg sodium acetate-3 hydrate;
0.16 μl acetic acid;
sodium hydroxide q.s. (i.e. to a final pH of) pH 5.5; and
water q.s (i.e. to a final volume of) 1 ml.

The antibody may be present at a concentration of 25±10 mg/ml, 120±20 mg/ml, or about 150±10 mg/ml.

In a further preferred embodiment, the invention provides a stable, aqueous pharmaceutical composition comprising:
25 mg/ml of an antibody as defined in claim 1;
20 mM sodium acetate;
150 mM sodium chloride;
sodium hydroxide q.s. (i.e. to a final pH of) pH 5.5
≧95% purity

It is appreciated that the pharmaceutical composition may also comprise a preservative. A “preservative” is a compound which can be included in the formulation to essentially reduce bacterial action therein, thus facilitating the production of a multi-use formulation, for example. Examples of potential preservatives include octadecyldimethylbenzyl ammonium chloride, hexamethonium chloride, benzalkonium chloride (a mixture of alkylbenzyldimethylammonium chlorides in which the alkyl groups are long-chain compounds), and benzethonium chloride. Other types of preservatives include aromatic alcohols such as phenol, butyl and benzyl alcohol, alkyl parabens such as methyl or propyl paraben, catechol, resorcinol, cyclohexanol, 3-pentanol, and m-cresol. A preferred preservative is benzyl alcohol. However, it is appreciated that a preservative may not be required since, in Example 3, the formulations have been shown to be sterile and free of bacterial and fungal contamination.

It is appreciated that the pharmaceutical composition may, in certain embodiments, also comprise a polyol. A “polyol” is a substance with multiple hydroxyl groups, and includes sugars (reducing and nonreducing sugars), sugar alcohols and sugar acids. Typical polyols have a molecular weight which is less than about 600 kD (e.g. in the range from about 120 to about 400 kD). A “reducing sugar” is one which contains a hemiacetal group that can reduce metal ions or react covalently with lysine and other amino groups in proteins and a “non-reducing sugar” is one which does not have these properties of a reducing sugar. Examples of reducing sugars are fructose, mannose, maltose, lactose, arabinose, xylose, ribose, rhamnose, galactose and glucose. Non-reducing sugars include sucrose, trehalose, sorbose, melezitose and raffinose. Xylitol, erythritol, threitol, sorbitol and glycerol are examples of sugar alcohols. Non-reducing sugars such as sucrose and trehalose may in certain circumstances be preferred polyols.

It is further appreciated that the pharmaceutical composition may, in certain embodiments, also comprise a surfactant, many of which are well known in the art. Exemplary surfactants include poloxamers (e.g. poloxamer 188). The amount of surfactant that may be added is such that it reduces aggregation of the antibody and/or minimises the formation of particulates in the formulation. For example, the surfactant may be present in the formulation in an amount from about 0.001% to about 0.2%, preferably from about 0.01% to about 0.1%.

In one embodiment, the composition does not comprise a polyol and/or a surfactant.

In an embodiment of the invention the pharmaceutical composition does not comprise an additive selected from polysorbate 20, arginine, histidine, glutamic acid and mannitol.

In an embodiment, the invention provides a pharmaceutical composition wherein the antibody is provided at a purity of 95% or more (for example, 96% or 97% or 98% or 99% or more, such as 100%).

The pharmaceutical composition to be used for in vivo administration to a patient is preferably sterile. This is readily accomplished, for example, by filtration through a 0.22 μm sterile filter.

The pharmaceutical composition may be formulated for subcutaneous or intravenous administration. In certain embodiments, especially when formulated for intravenous administration, it is preferred that the pharmaceutical composition is isotonic. By “isotonic” we mean that the formulation of interest has essentially the same osmotic pressure as human blood. Isotonic formulations will generally have an osmotic pressure from about 250 to 350 mOsm. Isotonicity can be measured using a vapour pressure or ice-freezing type osmometer, for example.

In an embodiment, the pharmaceutical composition comprising the antibody has not been subject to prior lyophilisation.

Methods for making antibodies, such as an antibody having an antibody having a heavy chain amino acid sequence of SEQ ID No: 3 and a light chain amino acid sequence of SEQ ID No: 4 are very well known in the art.

In brief, for recombinant production of antibody 2D03, polynucleotides encoding it (FIG. 1) are inserted into replicable vectors for expression. Many suitable expression vectors are available. The vector components generally include a signal sequence, an origin of replication, one or more marker genes, an enhancer element, a promoter, and a transcription termination sequence.

Suitable host cells for the expression of glycosylated antibody are derived from multicellular organisms. Although plant and insect cells may be suitable host cells, it is preferred that the host cells are vertebrate cells, and propagation of vertebrate cells in tissue culture has become a routine procedure. Examples of useful mammalian host cell lines are COS-7, CV1, VERO-76, HEK293, BHK, CHO, TM4, HELA, MDCK, BRL 3A, W138, Hep G2, MMT, TRI, MRC5, NSO and FS4.

Host cells are transfected with expression vectors for antibody production and cultured in conventional nutrient media, modified as appropriate for inducing promoters, selecting transfectants, or amplifying the genes encoding the antibody sequences. The host cells used to produce the antibody may be cultured in a variety of well known and commercially available media which may be supplemented, as necessary, with hormones and/or other growth factors, salts, buffers, nucleotides, antibiotics, trace elements and energy sources such as glucose. Any other necessary supplements may also be included at appropriate concentrations that would be known to those skilled in the art. The culture conditions, such as temperature, pH and the like, are also well known in the art.

When using recombinant techniques, the antibody can be produced intracellularly in the periplasmic space or directly secreted into the medium. If the antibody is produced intracellularly, as a first step the particulate debris, either host cells or lysed cells, is removed, for example, by centrifugation or ultrafiltration. Where the antibody is secreted into the medium, supernatants from such expression systems are generally concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxylapatite chromatography, gel electrophoresis, dialysis, and affinity chromatography, with affinity chromatography being the preferred purification technique. The suitability of protein A as an affinity ligand depends on the species and isotype of any immunoglobulin Fc domain that is present in the antibody. Protein A can be used to purify antibodies that are based on human γ1, γ2, or γ4 heavy chains (Lindmark et al, (1983) J. Immunol. Meth. 62: 1-13). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Other useful techniques for protein purification include fractionation on an ion-exchange column, ethanol precipitation, Reverse Phase HPLC, chromatography on silica, chromatography on heparin Sepharoset™ chromatography on an anion or cation exchange resin (such as a polyaspartic acid column), chromatofocusing, SDS-PAGE, and ammonium sulfate precipitation.

Preferably, the antibody 2D03 which is formulated is essentially pure and desirably essentially homogeneous (i.e., free from contaminating proteins). By an “essentially pure” antibody formulation we mean a composition comprising at least 90% by weight of the antibody, based on the total weight of proteins in the composition, and preferably at least 95% by weight. An “essentially homogeneous” antibody formulation means a composition comprising at least 99% by weight of antibody, based on total protein weight in the composition.

A second aspect of the invention provides an article of manufacture comprising a sterile container containing the stable, aqueous pharmaceutical formulation as defined in the first aspect of the invention. The article of manufacture may be a single-use disposable syringe, a bottle or a vial, or the like. The container may be formed from a variety of materials such as glass or plastic. An exemplary container is a 3-20 ml single use glass vial. Alternatively, the container may be 3-100 ml glass vial. The container holds the composition, and optionally a label on, or associated with, the container may indicate directions for use. The article of manufacture may further include other materials desirable from a commercial and user standpoint, including other buffers, diluents, filters, needles, syringes, and package inserts with instructions for use.

As has been described in WO 2004/030607 and WO 2007/025781, antibody 2D03 has the ability to both prevent, and induce the regression of, atherotic plaques. Accordingly, a third aspect of the invention provides a method of treating and/or preventing and/or reducing and/or combating atherosclerosis, or a cardiovascular disease associated with atherosclerosis, in a patient, the method comprising administering a therapeutically effective amount of a pharmaceutical composition as defined above with respect to the first aspect of the invention to a patient in need thereof.

In the context of the present invention, a “therapeutically effective amount” of an antibody refers to an amount effective in the prevention or treatment of atherosclerosis, or a cardiovascular disease associated with atherosclerosis.

The invention includes the use of 2D03, i.e. an antibody having a heavy chain amino acid sequence of SEQ ID No: 3 and a light chain amino acid sequence of SEQ ID NO: 4, in the manufacture of a pharmaceutical composition as defined in the first aspect of the invention for treating and/or preventing and/or reducing and/or combating atherosclerosis, or a cardiovascular disease associated with atherosclerosis, in a patient.

The invention also includes a pharmaceutical composition as defined above in the first aspect of the invention for use in treating and/or preventing and/or reducing and/or combating atherosclerosis, or a cardiovascular disease associated with atherosclerosis, in a patient.

In an embodiment, the antibody in the pharmaceutical composition reduces the formation of atherosclerotic plaques in the patient, i.e. slows the development of atherosclerosis, and preferably reduces or prevents the formation of new atherosclerotic plaques.

In another embodiment, the antibody in the pharmaceutical composition induces regression of pre-existing atherosclerotic plaques in the patient.

By “regression of atherosclerotic plaques” we include the meaning of reducing the size and/or amount and/or extent of atherosclerotic plaques. Typically, regression of atherosclerotic plaques leads to a reduction in the area of the interior arterial surface covered by plaques. Thus by “regression of atherosclerotic plaques” we include reducing the overall plaque burden in the individual, as well as reducing the size of some, or all, of the individual atherosclerotic plaques. Regression of atherosclerotic plaques also leads to an increase in the vascular lumen (i.e. an increase in the effective cross-section of the arterial vessel) contributing to increased blood flow.

Methods for measuring the size and/or amount and/or extent of atherosclerotic plaques in an individual are well known to the person of skill in the art and include angiography, vascular ultrasound, computer tomography and magnetic resonance imaging.

By “reducing the size and/or amount and/or extent” we include a reduction of about 1-25%, such as a reduction of about 1 or 2 or 3 or 4 or 5%, or a larger reduction of about 6 or 7 or 8 or 9 or 10%, or a reduction of 10-25%. More preferred is a larger reduction of 25-50%, or 50-75%, or more.

By reducing the area of the interior arterial surface covered by atherosclerotic plaques we include a reduction of about 1-25%, such as a reduction of about 1 or 2 or 3 or 4 or 5%, or a larger reduction of about 6 or 7 or 8 or 9 or 10%, or a reduction of 10-25%. More preferred is a larger reduction of 25-50%, or 50-75%, or more.

By an increase in the effective cross-section of the arterial vessel we include the meaning of an increase of 1-25%, such as an increase of about 1 or 2 or 3 or 4 or 5%, or a larger increase of about 6 or 7 or 8 or 9 or 10%, or an increase of 10-25%. More preferred is a larger increase of 25-50%, or 50-75%, or 75-100%. Most preferably, the effective cross-section of the arterial vessel is increased 2- or 3- or 4- or 5- or 10-fold, or more. Clearly, the extent of increase of cross-section of the arterial vessel is dependent upon the level of arterial blockage caused by atherosclerotic lesions prior to treatment.

The atherosclerotic plaques to be regressed are typically those in the aorta of the individual, but may also be found in other arterial sites in the patient like the femoral, carotid and coronary arteries.

Typically, the patient to be treated is a mammal, including humans, domestic and farm animals, and zoo, sports, or pet animals, such as dogs, cats, horses, cows, sheep, pig, camel, etc. Preferably, the patient is a human.

Typically, the patient is a human patient who has atherosclerosis. It is appreciated that since the antibody present in the pharmaceutical composition leads to a reduction in the size of pre-existing atherosclerotic plaques (WO 2007/025781), the pharmaceutical composition is particularly useful for treating patients with advanced or severe atherosclerosis, and advanced or severe forms of a cardiovascular disease associated with atherosclerosis.

The patient may be a human patient who has, or is at risk of having, a cardiovascular disease associated with atherosclerosis. The term “cardiovascular disease associated with atherosclerosis” includes references to diseases that are medically linked to atherosclerosis in that they are a consequence of atherosclerotic lesions. Cardiovascular diseases associated with atherosclerosis that may be mentioned include coronary artery disease, myocardial infarction and strokes.

It is also appreciated that since the antibody in the pharmaceutical composition both reduces the formation of atherosclerotic plaques and induces the regression of pre-existing atherosclerotic plaques, the pharmaceutical composition is useful for reducing the risk of a cardiovascular disease associated with atherosclerosis in a patient who is at risk of developing said cardiovascular diseases due to the presence of the atherosclerotic plaques. The patient who is at risk of a cardiovascular disease associated with atherosclerosis may be one who has blood cholesterol levels that are likely to cause or exacerbate cardiovascular disease or dysfunction.

The patient may be one who is at risk of developing coronary heart disease because of multiple risk factors (including obesity, smoking, hypertension, diabetes mellitus and a family history of premature coronary heart disease); one with a familial condition characterised by very high plasma concentrations of cholesterol and/or triglycerides; one with hyperlipidemia not secondary to underlying diseases (such as hypothyroidism, nephrotic syndrome, hepatic disease or alcoholism); one with elevated LDL-cholesterol; or one under dietary hypolipidemic intervention (complementary treatment).

In an embodiment of this aspect of the invention, the invention may include the prior step of determining the size and/or amount and/or extent of atherosclerotic plaques in the individual. This may be done to assess whether the individual is in need of treatment to reduce his atherosclerotic plaque burden, or to provide a baseline measurement to assess the efficacy of such treatment, or for both purposes. It is appreciated that an atherosclerotic plaque burden in need of reduction may be due to the size and/or extent of the overall plaque burden. Additionally or alternatively, this could be due to the nature of the plaques, for example, how unstable they are.

Optionally, and typically, the invention may also comprise the subsequent step of determining the size and/or amount and/or extent of atherosclerotic plaques in the patient after the administration of the pharmaceutical composition, so as to assess the efficacy of the treatment in comparison with a baseline measurement taken prior to treatment.

Whether or not a particular patient is one who is expected to benefit from treatment may be determined by the physician.

Statins (inhibitors of 3-hydroxy-3-methylglutaryl-coenzyme A (HMG-CoA) reductase) have proved to be effective in preventing acute cardiovascular events by reducing plasma cholesterol content (and by additional mechanisms yet to be clarified). Administration of a statin in conjunction with the immunotherapy described above may be a useful means of treatment to supplement the regression of atherosclerotic plaques.

Accordingly, a fourth aspect of the invention provides a kit of parts comprising the components: a pharmaceutical composition, or a lyophilised composition, as defined above with respect to the first aspect of the invention, and a statin. Suitable and preferred statins are selected from atorvastatin, cerivastatin, fluvastatin, lovastatin, mevastatin, pravastatin, rosuvastatin and simvastatin. Typically, the statin is formulated for oral administration.

The components are each provided in a form that is suitable for administration in conjunction with the other. By “in conjunction” we include the meaning that the components may be suitable for simultaneous or combined administration to the patient. However, since the components are typically administered by different routes, by “in conjunction” we also include the meaning of consecutive administration or separate administration within the same treatment regime.

The kit may further include other materials desirable from a commercial or user standpoint, including other buffers, diluents, filters, needles, syringes, and package inserts with instructions for use. Preferably the two components in the kit are chosen to have a synergistic effect.

A fifth aspect of the invention includes a method of treating and/or preventing and/or reducing and/or combating a cardiovascular disease associated with atherosclerosis, the method comprising administering to the individual a pharmaceutical composition as defined above with respect to the first aspect of the invention and a statin.

The invention includes a pharmaceutical composition as defined above with respect to the first aspect of the invention and a statin for use in combination in treating and/or preventing and/or reducing and/or combating a cardiovascular disease associated with atherosclerosis.

Preferably the two dosage regimes are chosen to have a synergistic effect.

Suitable and preferred statins include atorvastatin, cerivastatin, fluvastatin, lovastatin, mevastatin, pravastatin, rosuvastatin and simvastatin.

All of the documents referred to herein are incorporated herein in their entirety by reference. In particular, the entire disclosures of WO 2004/030607 and WO 2007/025781 relating to antibody 2D03 are incorporated herein, in their entirety, by reference.

The listing or discussion of a prior-published document in this specification should not necessarily be taken as an acknowledgement that the document is part of the state of the art or is common general knowledge

The invention will now be described in more detail by reference to the following Examples and Figures.

FIG. 1 depicts a polynucleotide sequence encoding the 2D03 heavy chain (SEQ ID No: 1) and light chain (SEQ ID No: 2).

FIG. 2 depicts the amino acid sequence of the 2D03 heavy chain (SEQ ID No: 3) and light chain (SEQ ID No: 4), encoded by the polynucleotides depicted in FIG. 1. The CDRs are underlined.

### EXAMPLE 1

### Effect of pH on Stability

During development of the purification process we found that antibody 2D03 displayed different solubility characteristics compared to previous n-CoDeR® generated products. Typically, a 10-20 mM phosphate buffer, containing 150 mM Nacl, pH 7-7.5, results in a stable formulation of n-CoDeR® generated antibody products. However, antibody 2D03 was not stable in this formulation. Accordingly, we assessed the effect of pH and salt concentration on the stability of antibody 2D03.

In summary:
2D03 antibody product aggregated at pH above 6.0, but not at pH values below this.
concentration or buffer exchange by ultrafiltration generated aggregates when performed in solutions with low conductivity, less than 100 mM NaCl equivalent (in milliSiemens), but not at concentrations above this.

Initial studies showed that the antibody 2D03 product could be concentrated to at least 160 mg/ml if the pH was maintained at pH 5.5. and 150 mM NaCl was included.

### EXAMPLE 2

### Stability Tests

### Abstract

The aim of this study was to identify a stable formulation for antibody 2D03 with a concentration above 100 mg/ml. The stability of six different formulations with antibody concentrations between 100-150 mg/ml and pH 5.5 were investigated after incubation at 5° C. and 24° C. (accelerated study).

### Background

In Example 1 we found that antibody 2D03 displayed different solubility characteristics compared to previous n-CoDeR® generated antibody products. In summary, the product appeared to aggregate at pH above 6.0 and concentration or buffer exchange by ultrafiltration generated aggregates if performed in solutions with low conductivity. Based on these findings, the following formulation stability studies were limited to formulations at pH 5.5, with 150 mM NaCl included in all formulations.

Based on our previous experience with high-concentration n-CoDeR® product formulations, polysorbate 20 was included in most test formulations as this has been shown to enhance the stability of n-CoDeR® antibody formulations; arginine and glutamic acid were included as excepients in one formulation based on findings reported in Golovanov et al (2004) J. Am. Chem. Soc., 126: 8933-8939; mannitol was included in one test formulation as it has been shown to enhance the stability of antibody formulations; and histidine was tested as it is a generally suitable biological buffer with an appropriate buffering range.

### Analytical Methods

The following methods were used to assess the stability of antibody 2D03 in the different formulations. Each analysis (concentration, appearance, purity, antigen binding activity, osmolality and qualitative evaluation) was performed on each of Formulations I-VI after 0, 4, 8, 14 and 18 weeks of storage at both 5° C. and 24° C.

### Purity by Size-Exclusion Chromatography

The purity of the antibody was qualitatively determined by HPLC using a TSKgel 3000SWXL column from TOSOH Bioscience. This is a size exclusion column which separates molecules according to their molecular weight, and which has its most effective resolution between 10-500 kDa. A mobile phase of 5 mM potassium phosphate containing 0.4 M sodium chloride, pH 7.2 was used at a flow rate of 1 ml/min. UV-detection was performed at 280 nm, and the area of the monomer peak was calculated as a percent of the total peak area. Integration was automated following manually set parameters.

### Protein Concentration by A₂₈₀

Determination of protein content is based on the ultraviolet absorbance of proteins in the aromatic region at 280 nm. Absorbance at 280 nm is directly proportional to protein concentration according to Lambert-Beers law. The absorbance was determined using an Ultrospec 110 pro spectrophotometer. The sample was diluted in 10 mM sodium phosphate buffer, 0.15 M NaCl, pH 7.4 to give 0.05-1.0 AU. The protein concentration was calculated:

A=εbc

A is absorbance, ε is the extinction coefficient (in this case 1.62), c is concentration in mg/ml and b is the path length of the cuvette in centimetres.

### Cation-Exchange Chromatography

A qualitative evaluation of the antibody was performed by using cation-exchange chromatography which separates molecules based on differences between the overall charges of the protein. A ProPac® weak cation-exchange column was used. This column is specifically designed to provide high-resolution and high efficiency separations of proteins and glycoproteins with pI=3-10 and MW>10 000. 10 mM sodium acetate, pH 5.0 (mobile phase A) and 10 mM sodium acetate, 1 M NaCl, pH 5.0 (mobile phase B) were used at a flow rate of 1 ml/min. A linear gradient of 0-75% mobile phase B for 10 min was applied. Detection was performed by absorbance at 280 nm.

### Light Scattering at 410 nm

To estimate precipitation, light scattering was measured at 410 nm, using an Ultrospec 110 pro spectrophotometer. Deionised water was used for calibration.

### SDS-PAGE

SDS-PAGE Phast system™ was used according to the manufacturer's manual. The proteins (Phastgel Gradient 8-25) were detected by Comassie staining. The sample load concentration was about 0.5 mg/ml, and 3-4 μL was loaded per well. Fermentas PageRuler Prestained Protein Ladder was loaded in the first well of each gel.

### Antigen Binding

Microtiter plates were coated with MDA-ApoB100 overnight. After washing, the plates were blocked with 0.45% fish gelatin for one hour at room temperature. Standard 2D03 titrations and test samples were added to the coated and blocked microtiter plates and incubated at room temperature for two hours. After washing, P214 rabbit-anti-Human Ig-HRP conjugate was added and the plates incubated for another hour at room temperature. The binding was visualised with OPD-substrate (o-phenylenediamine dihydrochloride), and stopped with 1M HCl. The absorbance was read on a Versamax reader at two wavelengths, 490 nm (λₜₑₛₜ) and 650 nm (λ_{ref}). Data was collected by the SOFTmax Pro 4.0 software with which the calculation of specific antibody concentrations was performed. The antigen binding was calculated as specific concentration received by this ELISA method divided by received value from total protein analysis (measured by A₂₈₀).

### Osmolality

The quantitative osmolality was established, using a Micro-Osmometer Typ 13/13 DR. Calibration points are 0 and 300 mOsm/kg H₂0. The sample was diluted with Milli-Q H₂0 to fit the calibration interval, if needed.

### Purity

The purity of the formulations, measured at the end of the stability test time, was less than 95%

### Results

### Formulation I

| | | |
|---|---|---|
| Antibody 2D03 | 136 | mg/ml |
| Sodium acetate | 20 | mM |
| NaCl | 150 | mM |

| | |
|---|---|
| pH | 5.5 |

### Results

Stability at 5° C.: Stable at 14 weeks, loss of stability by 18 weeks

Stability at 24° C.: Stable at 8 weeks, loss of stability by 14 weeks

### Formulation II

| | | |
|---|---|---|
| Antibody 2D03 | 136 | mg/ml |
| Sodium acetate | 20 | mM |
| NaCl | 150 | mM |

| | |
|---|---|
| Polysorbate 20 | 0.1% (1.1 mg/ml) |
| pH | 5.5 |

### Results

Stability at 5° C.: Stable at 4 weeks, loss of stability by 8 weeks

Stability at 24° C.: Stable at 0 weeks, loss of stability by 4 weeks

### Formulation III:

| Antibody 2D03 | 136 | mg/ml |
|---|---|---|
| Sodium acetate | 20 | mM |
| NaCl | 150 | mM |
| Polysorbate 20 | 0.1% (1.1 mg/ml) | |
| Mannitol | 50 | mM |

| | |
|---|---|
| pH | 5.5 |

### Results

Stability at 5° C.: Stable at 8 weeks, loss of stability by 14 weeks

Stability at 24° C.: Stable at 8 weeks, loss of stability by 14 weeks

### Formulation IV:

| | | |
|---|---|---|
| Antibody 2D03 | 136 | mg/ml |
| Sodium acetate | 20 | mM |
| NaCl | 150 | mM |
| His-HCl | 50 | mM |

| | |
|---|---|
| Polysorbate 20 | 0.1% (1.1 mg/ml) |
| pH | 5.5 |

### Results

Stability at 5° C.: Stable at 0 weeks, loss of stability by 4 weeks

Stability at 24° C.: Stable at 0 weeks, loss of stability by 4 weeks

### Formulation V:

| | | |
|---|---|---|
| Antibody 2D03 | 136 | mg/ml |
| Sodium acetate | 20 | mM |
| NaCl | 150 | mM |
| Arginine | 50 | mM |
| Glutamate | 50 | mM |

| | |
|---|---|
| Polysorbate 20 | 0.1% (1.1 mg/ml) |
| pH | 5.5 |

### Results

Stability at 5° C.: Stable at 0 weeks, loss of stability by 4 weeks

Stability at 24° C.: Stable at 0 weeks, loss of stability by 4 weeks

### Formulation VI:

| | | |
|---|---|---|
| Antibody 2D03 | 151 | mg/ml |
| Sodium acetate | 20 | mM |
| NaCl | 150 | mM |
| His-HCl | 50 | mM |

| | |
|---|---|
| Polysorbate 20 | 0.1% (1.1 mg/ml) |
| pH | 5.5 |

### Results

Stability at 5° C.: Stable at 0 weeks, loss of stability by 4 weeks

Stability at 24° C.: Stable at 0 weeks, loss of stability by 4 weeks

### CONCLUSION

Formulation I was stable for at least 14 weeks at 5° C. and at least 8 weeks in the accelerated study at 24° C., and was the most stable formulation identified. This formulation was deemed to be unstable by 18 weeks at 5° C. and by 14 weeks at 24° C. due to the presence of precipitation as established by eye and by light scattering at 410 nm.

For each of formulations I to VI, no significant differences in purity, concentration, character or activity were detected by any of the methods described above in any of the formulations over time. The instability of all of the formulations was determined by precipitation as distinguished by eye and by light scattering at 410 nm.

### EXAMPLE 3

### Formulation of a Concentrated 2D03 Solution

### Summary

Antibody 2D03 in bulk solution (37.3 g in a volume of 1534.6 ml, based on a protein concentration of 24.3 mg/ml determined by A₂₈₀) was concentrated by filtration using a Pellicon filter (feed pressure: 1.4-2.1 bar; retentate pressure: 0-0.7 bar) to a final concentration of 120±20 mg/ml at room temperature (actual concentration 133 mg/ml). After concentration, the product was filtered through a sterile 0.22 μm filter and stored at +2° C. to +8° C. in sterile PETG bottles.

The concentrated antibody solution was assessed for appearance, protein concentration, pH, endotoxins, purity, isoelectric focusing, bioburden and antigen binding using recognised QC procedures as described below.

### Appearance

The sample solution was visually inspected for particles, clarity and colour. For examination of particles, the sample was examined in front of both black and white backgrounds. For determination of clarity, 1 ml of the sample was transferred to a glass tube and the opalescence examined against a black background, compared to reference solutions, according to European Pharmacopoeia 2.2.1. The sample was considered as clear if its clarity was the same as water or if its opalescence was not more pronounced than reference suspension I. If the sample did not meet these criteria, the level of opalescence was reported as less than the first reference suspension that is more opalescent than the sample, for example <II or <III. The colour of the sample was examined and compared to water against a white background. If the sample was as colourless as water it is reported as colourless. If not, the shade of colour was described.

The acceptance criterion was: practically free from particles. Opalescence and colour were reported.

### Protein Concentration by A₂₈₀

Protein concentration was determined as described in Example 2 except that a Shimandzu UV-1601 spectrophotometer was used.

The acceptance criterion was: protein concentration 120±20 mg/ml.

pH-Measurement

The pH meter, a combined pH Electrode, Radiometer-Copenhagen pHM-210, was calibrated in the range of measurement using standard buffer solutions from Radiometer Analytical. The pH of the sample was then measured.

The acceptance criterion was: pH 5.5±0.5.

### Endotoxins by Kinetic LAL

The Kinetic-QCL™ kit from BioWhittaker was used as a quantitative kinetic assay for the detection of gram-negative bacterial endotoxin. The assay is based on an enzymatic reaction where endotoxin activates a proenzyme in limulus amebocyte lysate (LAL), which in turn catalyses the cleavage of pNitroaniline (pNA), producing a yellow colour.

Samples and standard titrations of endotoxin were diluted in limulus amebocyte lysate (LAL) reagent water using endotoxin-free glass tubes. Dilutions of 100 μl were transferred to a pyrogen free 96-well plate. Each sample dilution was spiked with a known amount of endotoxin. The plate was pre-incubated in the Versamax microplate reader at 37° C. for 10 minutes. The LAL/substrate was dissolved in LAL reagent water. After the incubation, 100 μl of the LAL/substrate solution was added to each well. The reading was started immediately and monitored each 2.5 minutes for 40 readings. The reaction was conducted at 37° C. The absorbance was read at 405 nm. The time required for the yellow colour to appear is inversely proportional to the amount of endotoxin present. Data is collected by the SOFTmax Pro 4.0 software, where calculations are performed. The quantities of endotoxin are expressed in defined International Units as IU/ml. The lowest sample dilution giving a 50-150% recovery of the spiked endotoxin is accepted.

The acceptance criterion was: endotoxins ≦4.0 IU/ml.

### Purity (HPLC-SEC)

Using a Beckman System Gold system, the purity of the antibody was quantified by HPLC using a TSK3000 column (TosoHaas). This is a size exclusion column which separates molecules according to their molecular weight, and which has its highest resolution between 10 to 500 kDa. A mobile phase of 5 mM potassium phosphate containing 0.4 M sodium chloride, pH 7.2 was used at a flow rate of 1 ml/min. The injection volume was 20 μl. UV-detection was performed at 280 nm. The integrated surface area was automatically calculated using manually set parameters and the software 32 Karat Version 5.0.

The acceptance criterion was: ≧90 area %. Retention time and area % for main peak and peaks ≧0.5 area % were reported.

### Purity (SDS-PAGE)

The purity of the sample was assessed by SDS-polyacrylamide gel electrophoresis. Samples containing protein were heated at 95° C. for five minutes in Tris-Glycine-SDS-Bromophenolblue buffer with and without mercaptoethanol. Samples and molecular weight Broad Range (BioRad) standards were loaded on Pre-Cast Gels, 4-20% from Novex. The gels were run in Tris-Glycine-SDS buffer at 125V for 120 minutes using Novex X-cell II Mini cell equipment. Proteins were visualised by staining with GELCODE Blue stain. The stained gels were scanned with a BioRad GS-710 densitometer, and the relative purity calculated. The results were evaluated using Quantity-One software (BioRad).

The acceptance criteria were: Comparability to reference, and no additional bands corresponding to more than 10% of the total protein.

### Isoelectric Focusing

Isoelectric focusing was performed on a Multiphor II electrophoresis unit using IsoGel® Agarose Plates, pH range 3-10. The anode solution was 0.5 M Acetic acid and the cathode solution 1 M sodium hydroxide. IEF Calibration kit Broad pI (Amersham Biosciences) was used as a marker. Samples were desalted by dialysis against 1% glycine before application onto the gel. The temperature was set to 10° C. and the samples pre-focused at 1500 V and 150 mA. After pre-focusing, the effect was increased to 25 W and the focusing was done for ˜60 minutes. Proteins were detected with Coomassie staining and the gel was scanned with a GS-710 densitometer (BioRad). The Quantity One software calculated the pI-values of the samples using a calibration curve created from the migration distance of the marker proteins and their pI-values.

This method was optimised for the 2D03 antibody and we found that the optimal amount of protein to load was 25 μg at 5 cm distance from the cathode. Using these findings, the samples were assayed in triplicate. pI-values and the number of bands were determined, with results reported as pI (min)−pI (max) and number of bands.

The acceptance criterion was: Comparability to reference.

### Bioburden

Within 24 h after sampling, the product was incubated on a Tryptone Soya agar plate at 32.5±2.5° C. for detection of bacteria, and on a Sabouraud Dextrose agar plate at 22.5±2.5° C. for detection of fungi. After three days, the Tryptone Soya agar plate was inspected and colonies counted, and after five days, the Sabouraud Dextrose agar plate was inspected.

The acceptance criterion was: no bacterial or fungal growth.

### Antigen Binding

Antigen binding was determined as described in Example 2.

The acceptance criterion was: ≧50% of reference.

### Results

The 2D03 concentrated solution was formulated as follows (per ml):
133 mg of antibody 2D03;
8.77 mg sodium chloride;
2.35 mg sodium acetate-3 hydrate;
0.16 μl acetic acid;
sodium hydroxide q.s. (i.e. to a final pH of) pH 5.5; and
water q.s (i.e. to a final volume of) 1 ml.

Following testing in duplicate, the 2D03 concentrated solution fulfilled the acceptance criteria for all of the above tests.

### EXAMPLE 4

### Long-Term Stability Test of a 2D03 Preparation

Long term stability studies were performed to demonstrate that the antibody preparation had sufficient shelf-life as required for use in a clinical setting.

Antibody preparations containing 25 mg/ml 2D03 antibody in 20 mM sodium acetate, 150 mM sodium chloride, pH 5.5 were stored at +5° C. for 12 months. Purity of the preparations was above 95%.

| Test | Method | Acceptance criteria | Results 12 Months |
|---|---|---|---|
| Protein | Absorbance | 25.0 ± 5.0 mg/ml | 26.2 |
| concentration | at A280 | | |
| pH | pH meter | 5.5 ± 0.5 | 5.6 |
| Purity | HPLC-SEC | ≧95.0% | 96.8 |

Stability test and results:
These results demonstrate that the 2D03 antibody is sufficiently stable in this formulation for 12 months at +5° C. to allow for use in a clinical setting.

### EXAMPLE 5

### Long-Term Stability Test of a 2D03 Preparation

An antibody preparation as the one of Example 4 was stored at +25° C. for 6 months.

| Test | Method | Acceptance criteria | Results 6 Months |
|---|---|---|---|
| Protein | Absorbance at | 25.0 ± 5.0 mg/ml | 25.4 |
| concentration | A280 | | |
| pH | pH meter | 5.5 ± 0.5 | 5.6 |
| Purity | HPLC-SEC | ≧95.0% | 95.7 |

These results demonstrate that the 2D03 antibody is sufficiently stable in this formulation for 6 months at +25° C. to allow for use in a clinical setting.

1. An aqueous pharmaceutical composition comprising a therapeutically effective amount of an antibody having a heavy chain amino acid sequence of SEQ ID No: 3 and a light chain amino acid sequence of SEQ ID No: 4 and a pharmaceutically-acceptable adjuvant, diluent, carrier or excipient, wherein said composition has a pH of 4 to 6.

2. A pharmaceutical composition according to claim 1 wherein the composition has a pH of 4.5 or higher.

3. A pharmaceutical composition according to claim 2 wherein the composition has a pH of 4.9 or higher.

4. A pharmaceutical composition according to claim 3 wherein the composition has a pH of 4.9 to 5.1.

5. A pharmaceutical composition according to claim 4 wherein the composition has a pH of about 5.

6. A pharmaceutical composition according to claim 1 wherein the composition has a pH of 5 to 6.

7. A pharmaceutical composition according to claim 6 wherein the composition has a pH of 5 to 5.9.

8. A pharmaceutical composition according to claim 7 wherein the composition has a pH of 5.4 to 5.6.

9. A pharmaceutical composition according to claim 8 wherein the composition has a 30 pH of about 5.5.

10. A pharmaceutical composition of claim 1, wherein the antibody is provided at a purity of 95% or more.

11. A pharmaceutical composition of claim 1, wherein the antibody is present at a concentration of 10 to 200 mg/ml.

12. A pharmaceutical composition according to claim 11 wherein the antibody is present at a concentration of 5 25 to 150 mg/ml.

13. A pharmaceutical composition according to claim 11 wherein the antibody is present at 25±10 mg/ml.

14. A pharmaceutical composition according to claim 11 wherein the antibody is present at 120±20 mg/ml.

15. A pharmaceutical composition according to claim 11 wherein the antibody is present at about 150±10 mg/ml.

16. A pharmaceutical composition of claim 1, wherein the composition comprises an acetate buffer.

17. A pharmaceutical composition according to claim 16 wherein the acetate is present at 5-30 mM.

18. A pharmaceutical composition according to claim 17 wherein the acetate is present at 10-30 mM.

19. A pharmaceutical composition according to claim 18 wherein the acetate is present at about 20 mM.

20. A pharmaceutical composition of claim 1, wherein the composition comprises sodium chloride.

21. A pharmaceutical composition according to claim 20 wherein the sodium chloride is present at 100 mM to 200 mM.

22. A pharmaceutical composition comprising, per ml:
15 to 160 mg of an antibody as defined in claim 1;
8.77 mg sodium chloride;
2.35 mg sodium acetate-3 hydrate;
0.16 μl acetic acid;
sodium hydroxide q.s. pH 5.5; and
water q.s 1 ml.

23. A pharmaceutical composition according to claim 22 wherein the antibody is present at a concentration of 25±10 mg/ml, 120±20 mg/ml, or 150±10 mg/ml.

24. A pharmaceutical composition comprising:
25 mg/ml of an antibody as defined in claim 1;
20 mM sodium acetate;
150 mM sodium chloride;
sodium hydroxide q.s. pH 5.5.

25. A pharmaceutical composition of claim 24, wherein the composition further comprises a preservative.

26. A pharmaceutical composition according to claim 25 wherein the preservative is benzyl alcohol.

27. A pharmaceutical composition of claim 1, wherein the composition is formulated for subcutaneous or intravenous administration.

28. A pharmaceutical composition of claim 1, which is stable at a temperature of 2-8° C. for at least 14 weeks.

29. A pharmaceutical composition of claim 1, which is stable at a temperature of 2-8° C. for at least 12 months.

30. A pharmaceutical composition of claim 1, which is stable at a temperature of 2-8° C. for at least 1.5 or at least 3 years.

31. A pharmaceutical composition of claim 1, which is stable at a temperature of about 24° C. for at least 8 weeks.

32. A pharmaceutical composition of claim 1, which is stable following freezing and thawing of the composition.

33. A pharmaceutical composition of claim 32, wherein the antibody is not subject to prior lyophilisation.

34. An article of manufacture comprising a sterile container holding a stable aqueous pharmaceutical formulation of claim 1.

35. An article of manufacture according to claim 34 which is a disposable syringe.

36. A kit of parts comprising a stable pharmaceutical composition of claim 1 and a statin.

37. A kit of parts according to claim 36 wherein the statin is formulated for oral administration.

38. A kit of parts according to claim 36 wherein the statin is selected from atorvastatin, cerivastatin, fluvastatin, lovastatin, mevastatin, pravastatin, rosuvastatin and simvastatin.

39. A method of combating atherosclerosis, or a cardiovascular disease associated with atherosclerosis, in a patient, the method comprising administering a pharmaceutical composition as defined in claim 1 to a patient in need thereof.

40. A method according to claim 39 wherein the antibody reduces the formation of atherosclerotic plaques in the patient.

41. A method according to claim 39 wherein the antibody induces regression of pre existing atherosclerotic plaques in the patient.

42.-43. (canceled)

44. A method according to claim 39, wherein the patient is a human patient who has atherosclerosis.

45. A method according to claim 39, wherein the patient is a human patient who has, or is at risk of having, a cardiovascular disease associated with atherosclerosis.

46. A method according to claim 45, wherein the cardiovascular disease associated with atherosclerosis is selected from coronary artery disease, myocardial infarction and stroke.

47. A method of combating a cardiovascular disease associated with atherosclerosis, the method comprising administering to the individual a pharmaceutical composition as defined in claim 1 and a statin.

48. A pharmaceutical composition of claim 1 and a statin for use in combination in combating a cardiovascular disease associated with atherosclerosis.

49.-53. (canceled)
